(19) United States
(12) Patent Application Publication
(10) Pub. No.: US 2013/0245495 A1
(43) Pub. Date: Sep. 19, 2013

(54) FLOCKING SWAB FOR BIOLOGICAL SAMPLING QUANTITATIVELY AND METHOD FOR MAKING THE SAME

(71) Applicants: Ansheng Li, Nanjing (CN); Yaohua Wu, Nanjing (CN); Shuhua Tan, Nanjing (CN); Zhonghua He, Nanjing (CN); Chaochao Fan, Nanjing (CN)

(72) Inventors: Ansheng Li, Nanjing (CN); Yaohua Wu, Nanjing (CN); Shuhua Tan, Nanjing (CN); Zhonghua He, Nanjing (CN); Chaochao Fan, Nanjing (CN)

(21) Appl. No.: 13/989,056

(22) PCT Filed: Feb. 9, 2011
(86) PCT No.: PCT/CN11/00203

§ 371 Date: May 22, 2013

(30) Foreign Application Priority Data
Dec. 24, 2010 (CN) 201010604572.2

(51) Int. Cl. A61F 13/38

(52) U.S. Cl. CPC A61F 13/385
USPC 600/572; 427/462

(57) ABSTRACT

The invention relates to a flocking swab for biological sampling quantitatively and a method for making the same. One or two ends of the swab shaft form a loop by injection molding process. Flocking fiber is flocked into a layer on the surface of the loop by conventional electrostatic flocking process. By controlling the diameter of the loop frame, the diameter of the cylinder of the loop, and parameters of flocking fibers, a quantitative swab for biological sampling can be made. The swab disclosed in this invention can be used in quantitative sampling, has large sampling volume and securely attached swab tip. Moreover, it is safe and easy to use.

### TECHNOLOGY FIELD

The present invention relates to a research tool used in biological experiment and the method of making the same. Specially, it relates to a flocking swab that is used in quantitatively sampling of biological specimens and the method of making it.

### TECHNOLOGY BACKGROUND

In life science research, collecting and transporting biological specimens (cells and macromolecules) is important for any laboratory test. Of various sampling and transporting methods, swabbing is the most convenient and, therefore, the most frequently used. Swab is the most important component of the sampling process in which biological specimens are reliably collected at the sampling location and safely transported to laboratory for further test.

The conventional biological sampling swab widely used nowadays is comprised of a swab shaft and a swab tip. The cylinder-shaped swab shaft is normally made of materials with appropriate hardness and flexibility. One end of the shaft is typically to be held by hand, and the other end is available for the attachment of a swab tip. Sometimes, absorbent tips are attached on both ends of the shaft. Swab tip is used for picking up biological specimens and is usually made into a bud shape. Based on materials and technologies used in tip manufacturing, swab can be classified into three major types: winding swab (made with long fiber), flocking swab (made with short flocking fiber) and foaming swab (made with polyurethane foam). More specifically, winding swab is made by winding or swapping long fibers around the end of swab shaft. Flocking swab is made by electrostatically flocking short flock fibers to cover the surface of the shaft tip. The fibers used in flocking include natural fiber, synthetic fiber or any blend of these two types. Foaming swab is made with foaming technology using polyurethane and chemical foaming agents.

Among all three types of swabs, winding swab is the first invented. However, it is technically challenging to assure high level of performance consistency between different batches of winding swabs because of the difficulty in minimizing variations in several key technical parameters. These parameters are such as the property and amount of adhesive, fiber length and fineness, as well as the tightness in winding. The later-invented flocking swab and foaming swab have somewhat improved performance consistency, especially in specimen absorbing and releasing. In terms of the mechanisms of absorbance utilized by these types of swabs, winding swab and flocking swab utilize the surface absorption property of fiber, and foaming swab utilizes the surface adhesive property of micro-pores in foam. Foaming swab and flocking swab also utilize capillary effect for absorption.

Overall, despite the wide use of conventional swabs, all three types of swabs have following drawbacks:

(1) Flocking swab and foaming swab absorb fewer specimens than winding swab does.

(2) It is technically difficult to make thin shaft swabs. A thin shaft flocking swab renders inadequate surface for flocking fiber to be flocked at the tip. Moreover, for foaming swabs, swab tips normally do not attach to the shaft as tightly as in other types of swabs. With thin shaft, foaming swab tips are prone to detachment.

(3) The tip of flocking swab and foaming swab locates at the end of the shaft. This configuration is less effective in specimen absorption because, normally, only less than half of the surface of the tip could be in contact with the samples. It is an issue especially when working with liquid samples.

(4) None of three types of swab can be used in quantitative analysis. It is impossible to control the amount of samples in each swabbing with conventional swab manufacturing technology.

(5) The relatively sharp end of all three types of swab shaft poses potential injury hazard when used in picking up specimens on human body.

### BRIEF DESCRIPTION OF THE DRAWINGS

FIG. 1 shows a sectional view of the embodiment example 1 of this invention

FIG. 2 shows a sketch view of the cross-section of loop frame in embodiment example 1 of this invention.

FIG. 3 shows a section view of the loop structure in embodiment example 1 of this invention.

FIG. 4 shows a section view of a conventional swab made without using technologies described in this invention.

Figure details: (1) Swab shaft, (2) Flocking layer, (3) Pre-cut mick, (4) Loop frame.

### DETAILED DESCRIPTION

The main object of this invention is to overcome major drawbacks of conventional swabs in collecting biological specimens. These drawbacks include the lack of utility in quantitative analysis, low absorbing capacity, easy detachment of swab tips and potential safety hazard. Further object of this invention is to describe a manufacturing process in making such a quantitative flocking swab.

Specifically, for these objects, the invention describes a quantitative flocking swab for biological samples that is comprised of a swab shaft and a swab tip. One end or both ends of the swab shaft is made into the shape of a loop, on which a layer of fiber is to be flocked to form a swab tip.

The inner diameter of the loop is between 1.25 mm and 10 mm. The diameter of the cross section of the loop frame is between 0.2 mm and 3 mm. These configurations provide an effective surface area in the range of between 2.5 mm² and 296 mm² for flocking fiber.

The loop frame can be made in the shape of circular, oval, triangular or any polygonal with more than 3 edges. The cross section of the loop frame can be circular, oval or any polygonal.

The material used in making swab shaft can be polypropylene (PP), polyethylene (PE), polyethylene terephthalate (PET), polystyrene (PS) or other engineering plastics.

The fiber that covers the surface the loop frame to form flocking layer can be natural fiber, synthetic fiber or blend of these two kinds.

The length of the flocking fiber is 0.2-3.0 mm. The fineness of the flocking fiber is 0.33-7.0 dtex. The fiber density is 1.5-350 μg/mm².

Nylon fiber is used as flocking fiber to cover the surface of the loop frame.

The swab shaft can be made with high impact polystyrene material that can be easily broken up at any point, given certain pressure applied. When using other materials to make shafts, a pre-cut nick can be made in pre-determined location to assist breaking.

The manufacturing process of quantitative swab comprised of making of swab shaft and making of swab tip. Further details of the process are as follows: plastic injection molding is used to producing swab shaft with loop frame at one end or both ends. The loop provides a substrate for the flocking process in which the surface of the loop will be covered with a layer of flocking fiber with electrostatic technology. Natural fiber, synthetic fiber or any blend of these two fibers can be used in flocking. The effective area for flocking is determined by the shape and the size of the loop frame. The diameter of the loop frame can be made between 1.25 mm and 10 mm. The diameter of the cross-section of the loop cylinder can be made between 0.2 mm and 3 mm. The effective flocking surface of the swab shaft in this configuration is 2.5-296 mm². Swab tips made with this configuration have absorbing capacity of 10-200 μl.

The amount of liquid specimen to be picked up by a swab is determined by aforementioned parameters of loop frame and fiber density. A swab that is made with parameters within aforementioned ranges will consistently absorb a fixed volume between 10 μl and 200 μl when measured by weight using de-ionized water as at 25° C.

Additional parameters used in engineering are The length of the flocking fiber is 0.2-3.0 mm. The fineness of the flocking fiber is 0.33-7.0 dtex. The fiber density is 1.5-350 μg/mm².

An example of flocking fiber used is nylon fiber.

The amount of specimens a quantitative sampling flocking swab can pick up is determined by its effective flocking surface area, which, in turn, is determined primarily by the inner diameter of loop frame and diameter of the cross-section of the loop cylinder. In addition, fiber length, fiber fineness and fiber density all affect the volume. Compared to conventional bud-shaped swab tip, the swab described in this invention has a loop frame at the end which effectively increases the area for flocking fibers, and, as the result, has increased sampling capacity. Furthermore, the sampling amount of a swab in this invention can be precisely controlled by controlling the inner diameter of loop frame and diameter of the cross-section of the loop cylinder, as well as fiber length, fiber fineness and fiber density. A swab made in this process in test has been demonstrated to absorb the same quantity of water within the range of 10-200 μl in repeated experiments as measured by weight with water at 25° C.

Because the effective sampling part of this flocking swab as described in this invention locates at the tip, it forms sufficient contact with samples. Also because the flocking swab in this invention utilizes a loop frame, not the conventional bud structure, it minimizes the possibility of detachment and chance of injury hazards.

To summaries the features of a quantitative flocking swab for biological samples and the method of making the same:

(1) The sampling capacity of a quantitative flocking swab for biological samples in this invention can be custom designed according to the nature of sampling specimens and sampling needs.

(2) Swab tip of a quantitative flocking swab described in this invention is in complete contact with the specimens and maximizes sampling quantity.

(3) Quantitative flocking swabs for biological samples in this invention made with the same configuration pick up precisely the same amount of sample, a character that useful in quantitative assays.

(4) A quantitative flocking swab for biological samples in this invention minimizes the chance of tip detachment.

(5) A quantitative flocking swab for biological samples in this invention eliminates the chance of accidental injury hazards on sampling subjects.

### EMBODIMENT EXAMPLES

Several embodiments of this invention are given with reference to description of the drawings.

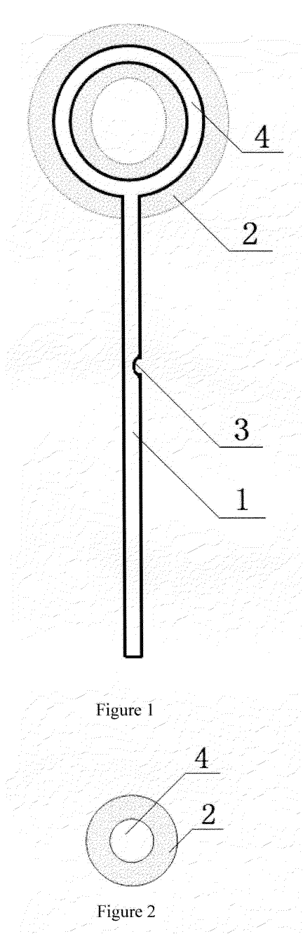

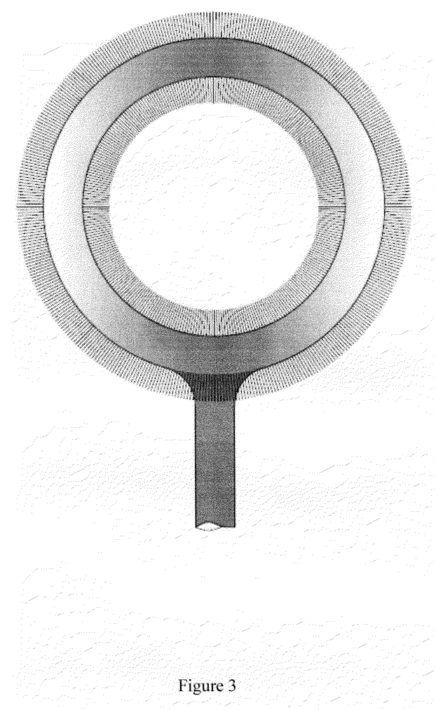

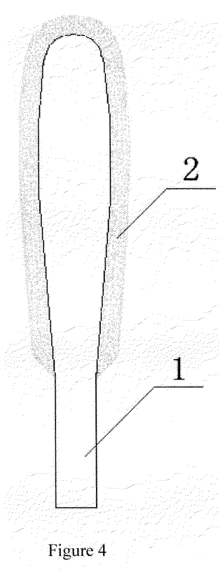

Embodiment example 1, given as illustrated with FIG. 1, FIG. 2 and FIG. 3, is to describe a quantitative sampling flocking swab for biological samples and the method of retaking the same, comprising swab shaft and swab tip. The method to produce such a swab comprises forming a loop frame (4) with plastic injection molding technology at the one end of swab shaft (1). Alternatively, both ends of the swab shaft (1) can be made into loop frames (4). In this embodiment, the method to produce such a swab comprises forming a circular loop frame. Alternatively, the shape of the loop can be made into shapes of oval, triangular or any polygonal with more than 3 edges. In this embodiment, the cross-section of the loop cylinder is circular. Alternatively, it can be made into oval or polygonal.

In this embodiment, the inner diameter of this loop frame is 3.91 mm, which is in the range of 1.25-10 mm, as described previously. In this embodiment, the diameter of the cross-section, of the loop cylinder is 1.19 mm, which is in the range of 0.2-3 mm, as described previously. In this embodiment, polyethylene (PE) is used to make swab shaft (1) and loop frame (4). Alternatively, polyethylene terephthalate (PET), polystyrene (PS) or other engineering plastics can be used. In this embodiment, a precut nick was made in the shaft (1). which is to assist breaking the shaft at this particular location with minimal force. Alternatively, high impact polystyrene material can be used so that at any point the swab shaft can be easily broken without the necessity of a precut nick.

In this embodiment, flocking fiber layer (2) is planted to cover the surface of the loop frame (4) using electrostatic flocking technology. The configuration of the loop structure (4) provides an effective planting surface area in the range of 2.5-296 mm². In this embodiment example, the effective surface area is 46 mm²; the length of flocking fiber in flocking layer (2) is between 0.2-3.0 mm. In this embodiment of example, it is 0.6 mm; the fineness of fiber in flocking layer (2) is between 0.33-7.0 dtex. The fiber fitness in this embodiment example is 1.5 dtex; the fiber density of flocking fiber in flocking layer (2) is between 1.5-350 μg/mm². In this example, it is 17 μg/mm². The fiber planted in the flocking layer comprising natural fiber, synthetic fiber or any blend of these two. The fiber used in this example is nylon fiber.

Compared to conventional swab showed in FIG. 4, the quantitative swab for biological sampling in this embodiment has several advantages. For instances, it can be made with simple manufacturing process. The swab has a large sampling capacity and precisely absorbs 55 μl of liquid samples (measured using water at 25° C.). In addition, swab tip is securely attached with the shaft which offers operational convenience and safety.

### Embodiment Example 2

In another preferred embodiment, example 2, a quantitative swab for biological samples has the fallowing configuration: the inner loop diameter is 5 mm; the diameter of cross-section of loop cylinder is 1.5 mm; a loop frame in this embodiment has an area of 74 mm² for flocking fiber planting. Moreover, the length of the fiber is 1.5 mm. The fineness of flocking fiber is 6.67 dtex. The fiber density is 328 μg/mm². The swab is manufactured in the same technology as example 1. A swab in this figuration absorbs precisely 110 μl sample solution (measured using water at 25° C.). All other swab parameters are the same as these in example 1.

### Embodiment Example 3

Example 3 is yet another preferred embodiment. A quantitative swab for biological samples has the following configuration: the inner loop diameter is 1.5 mm; the diameter of cross-section of loop is 0.5 mm; a loop frame in this embodiment has an area of 7.39 mm² for flocking fiber planting. Moreover, the length of the fiber is 1.0 mm. The fineness of flocking fiber is 1.5 dtex, the fiber density is 109 μg/mm². The swab is manufactured in the same technology as example 1. A swab in this figuration absorbs 20 μl sample solution (measured using water at 25° C.). All other swab parameters are the same as these in example 1.

What is claimed is:

1. A quantitative flocking swab for biological samples comprising:
a swab shaft (1) with one end or both ends forming a loop frame (4) and a swab tip that is formed by covering loop frame (4) with a layer of flocking material.

2. A quantitative flocking swab for biological samples as claimed in claim 1 wherein said loop frame (4) has an inner diameter between 1.25 mm and 10 mm. The diameter of the cross-section of said loop frame cylinder is between 0.2 mm and 3 mm, and the effective surface area for flocking fiber planting on the loop frame is between 2.5 mm² and 296 mm².

3. A quantitative flocking swab for biological samples as claimed in claim 2 wherein said loop frame (4) is one of the following shapes: circular, oval, triangular, or other polygonal with more than 3 edges. The cross-section of said loop cylinder is one of the following: circular, oval or polygonal;
Material used to make said swab shaft is one of the following materials: polypropylene (PP), polyethylene (PE), polyethylene terephthalate (PET), polystyrene (PS) or other engineering plastics;
The material in the flocking layer (2) to cover the said loop frame (4) is made of natural fiber, synthetic fiber or any blend of the these two kinds of fibers.

4. A quantitative flocking swab for biological samples as claimed in claim 1, 2, or 3 wherein a layer of said flocking material (2) has fiber length between 0.2-3.0 mm, fiber fineness between 0.33-7.0 dtex, and fiber density between 1.5-350 μg/mm².

5. A quantitative flocking swab for biological samples as claimed in claim 4 wherein the said flocking layer (2) which covers said loop frame (4) is made of nylon.

6. A quantitative flocking swab for biological samples as claimed in claim 3 wherein said shaft (1) has a pre-cut nick (3) to facilitate easy breaking or to be made with high impact polystyrene material that can be easily broken up at any point given certain pressure in which a pre-cut nick (3) is not necessary.

7. A method of making a quantitative flocking swab for biological samples as claimed in claim 1 comprising steps of making swab shaft and swab tip. Specifically, The method to produce such a swab comprises forming a loop frame (4) with plastic injection molding technology at the one end or both ends of swab shaft (1); a layer of flocking fiber (2) is planted to cover the surface of the loop frame (4) using electrostatic flocking technology to form a swab tip; the fiber planted in the flocking layer comprising natural fiber, synthetic fiber or any blend of these two; an elective planting surface area in the range of 2.5-296 mm² will be obtained by controlling the inner diameter of the loop frame (4) to be in the range of 1.25 mm-10 mm and the diameter of the cross-section of the loop cylinder to be in the range of 0.2-3 mm. A quantitative flocking swab for biological samples produced in this process has ability to sample biological specimens quantitatively in the range of 10-200 μl.

8. A method of making a swab for quantitative flocking swab for biological samples as claimed in claim 7 further comprising method for producing said swab with fiber length between 0.2-3.0 mm, fiber fineness between 0.33-7.0 dtex and fiber density between 1.5-350 μg/mm².

9. A method of making a quantitative flocking swab for biological samples as claimed in claim 7 further comprising steps of controlling structure of said loop frame (4) and density of said flocking layer so that precise amount of liquid samples can be picked up by said swab tip. Specifically, in testing using water measured at 25° C., each configuration of parameters corresponding to a unique value in the range of 10-200 μl.

10. A method of making a quantitative flocking swab for biological samples as claimed in claim 9 wherein said material is nylon.
